(19) United States
(12) Patent Application Publication
(10) Pub. No.: US 2026/0207962 A1
(43) Pub. Date: Jul. 23, 2026

(54) DEVICE AND METHOD FOR USING LIGHT THERAPY FOR MEDICAL TREATMENT

(71) Applicant: Healing Light Therapies, LLC, Englewood, OH (US)

(72) Inventor: John Prikkel, III, Englewood, OH (US)

(21) Appl. No.: 19/456,505

(22) Filed: Jan. 22, 2026

Related U.S. Application Data
(60) Provisional application No. 63/748,118, filed on Jan. 22, 2025.

(51) Int. Cl. A61N 5/06

(52) U.S. Cl. CPC A61N 5/0613; A61N 2005/0627; A61N 2005/0629; A61N 2005/0632; A61N 2005/0642; A61N 2005/0652; A61N 2005/0659; A61N 2005/0663

(57) ABSTRACT

A device for providing light therapy for patients comprises a base with posts and a visor including an array of light-emitting devices. The array of light-emitting devices includes a first cluster of the light-emitting devices and a second cluster of the light-emitting devices. Each cluster includes a set of light-emitting devices of a first color, a set of light-emitting devices of a second color, and a set of infrared (IR) light-emitting devices. A controller controls activation of the array of light-emitting devices such that the array of light-emitting devices is active for a first period of time. After the array of light-emitting devices is active for the first period of time, the array of light-emitting devices is inactive for a second period of time. After the array of light-emitting devices is inactive, the array of light-emitting devices activates for a third period of time.

### CROSS REFERENCE TO RELATED APPLICATIONS

This application claims the benefit of U.S. Provisional Patent Application Serial No. 63/748,118, filed January 22, 2025, having the title DEVICE AND METHOD FOR AIDING PATIENTS WITH MACULAR DEGENERATION, the entire disclosure of which is hereby incorporated herein by reference.

### BACKGROUND

Various aspects of the present disclosure relate generally to procedures to treat macular degeneration and more specifically to light-therapy procedures to treat macular degeneration.

Macular Degeneration, sometimes referred to as Age-Related Macular Degeneration (AMD or ARMD) or Macular Dystrophy, is a medical condition which may result in blurred or no vision in the center of the visual field. Over time, however, some people experience a gradual worsening of vision that may affect one or both eyes. While it does not result in complete blindness, loss of central vision can make it hard to recognize faces, drive, read, or perform other activities of daily life.

Forms of macular degeneration include "dry" and "wet" forms, with the dry form making up 90% of cases. The difference between the two forms is categorized by the change in the macula. Those with dry form AMD have drusen, cellular debris in their macula that gradually damages light-sensitive cells and leads to vision loss. In wet form AMD, blood vessels grow under the macula, causing blood and fluid to leak into the retina.

### BRIEF SUMMARY

According to aspects of the present disclosure, a device for aiding patients with macular degeneration comprises a base with posts and a visor including an array of light-emitting devices. The array of light-emitting devices includes a first cluster of the light-emitting devices and a second cluster of the light-emitting devices. Each cluster includes a set of light-emitting devices of a first color, a set of light-emitting devices of a second color, and a set of infrared (IR) light-emitting devices. A controller that controls activation of the array of light-emitting devices such that the array of light-emitting devices is active for a first period of time. After the array of light-emitting devices is active for the first period of time, the array of light-emitting devices is inactive for a second period of time. After the array of light-emitting devices is inactive for the second period of time, the array of light-emitting devices is active for a third period of time.

According to further aspects of the present disclosure, a process for controlling a visor for aiding users with macular degeneration comprises activating a first cluster of light-emitting devices and a second cluster of light-emitting devices for a first period of time. The first cluster of light-emitting devices includes a first set of light-emitting devices of a first color, a first set of light-emitting devices of a second color, and a first set of infrared (IR) light-emitting devices. Further, the second cluster of light-emitting devices includes a second set of light-emitting devices of the first color, a second set of light-emitting devices of the second color, and a second set of infrared (IR) light-emitting devices. After the first cluster of light-emitting devices and the second cluster of light-emitting devices have been activated, the first cluster of light-emitting devices and the second cluster of light-emitting devices are deactivated for a second period of time. After the first cluster of light-emitting devices and the second cluster of light-emitting devices have been deactivated, the first cluster of light-emitting devices and the second cluster of light-emitting devices are activated for a third period of time.

According to yet further aspects of the present disclosure, a visor including an array of light-emitting devices. The array of light-emitting devices includes a first cluster of the light-emitting devices and a second cluster of the light-emitting devices. Each cluster includes a set of light-emitting devices of a first color, a set of light-emitting devices of a second color, and a set of infrared (IR) light-emitting devices. A controller that controls activation of the array of light-emitting devices such that the array of light-emitting devices is active for a first period of time. After the array of light-emitting devices is active for the first period of time, the array of light-emitting devices is inactive for a second period of time. After the array of light-emitting devices is inactive for the second period of time, the array of light-emitting devices is active for a third period of time.

Embodiments of the visor include a communications port that allows the visor to be part of a modular system that allows an ancillary device to be communicably coupled to the visor. For example, the ancillary device may be a device that includes several arrays or clusters of light-emitting devices that provide a light (visible, infrared, etc.) to a patient’s forehead, where operation of the ancillary device is provided by the controller in the visor.

### BRIEF DESCRIPTION OF THE SEVERAL VIEWS OF THE DRAWINGS

FIG. 1 is a drawing of an isometric view of a device for aiding people with macular degeneration, according to aspects of the present disclosure;

FIG. 2 is a drawing of a front view of a device for aiding people with macular degeneration, according to aspects of the present disclosure;

FIG. 3 is a drawing of a side view of a device for aiding people with macular degeneration, according to aspects of the present disclosure;

FIG. 4 is a drawing of a top view of a device for aiding people with macular degeneration, according to aspects of the present disclosure;

FIG. 5 is a drawing of a front view of a visor for a device for aiding people with macular degeneration, according to aspects of the present disclosure;

FIG. 6 is a flow chart illustrating a process for operating a device for aiding people with macular degeneration, according to aspects of the present disclosure;

FIG. 7 is a diagram of an ancillary device that couples to the visor via a communications port for use in light therapy on a patient’s forehead, according to aspects of the present disclosure.

### DETAILED DESCRIPTION

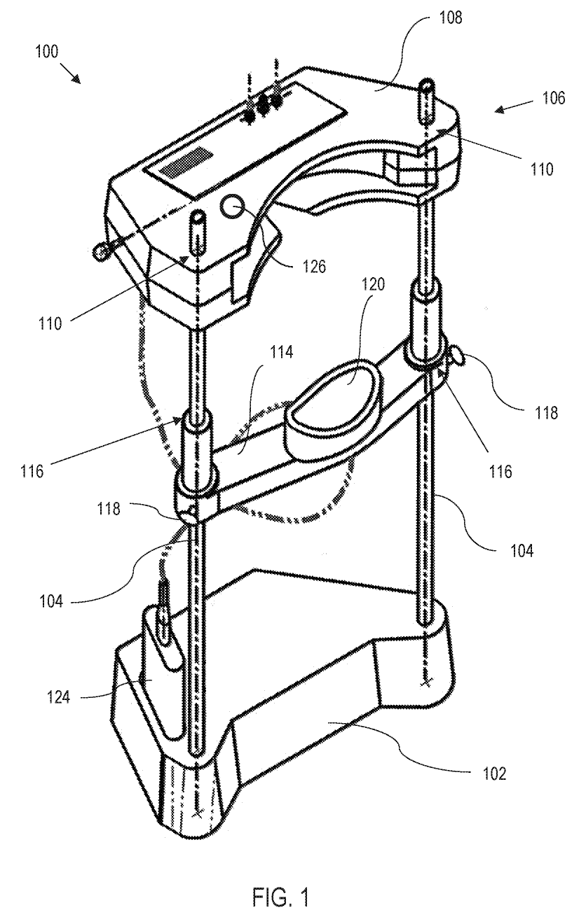

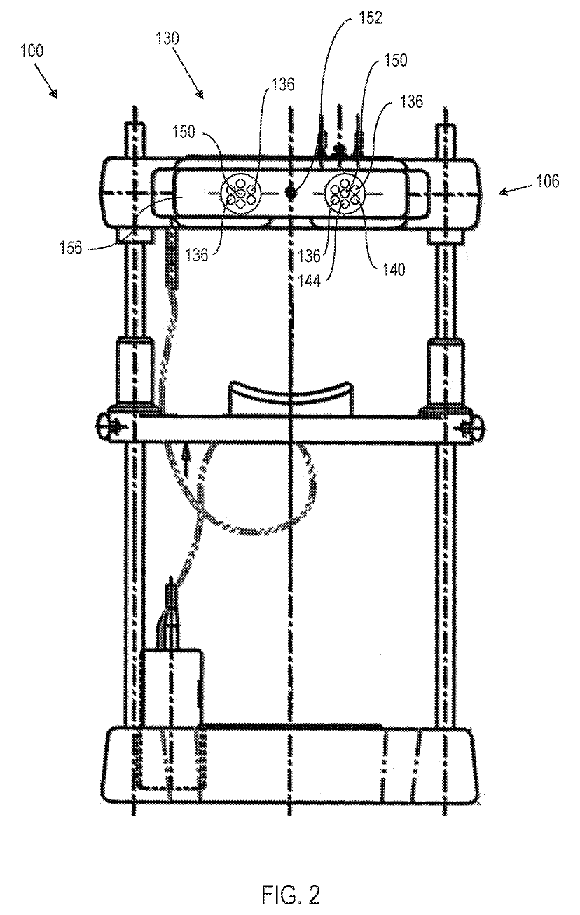

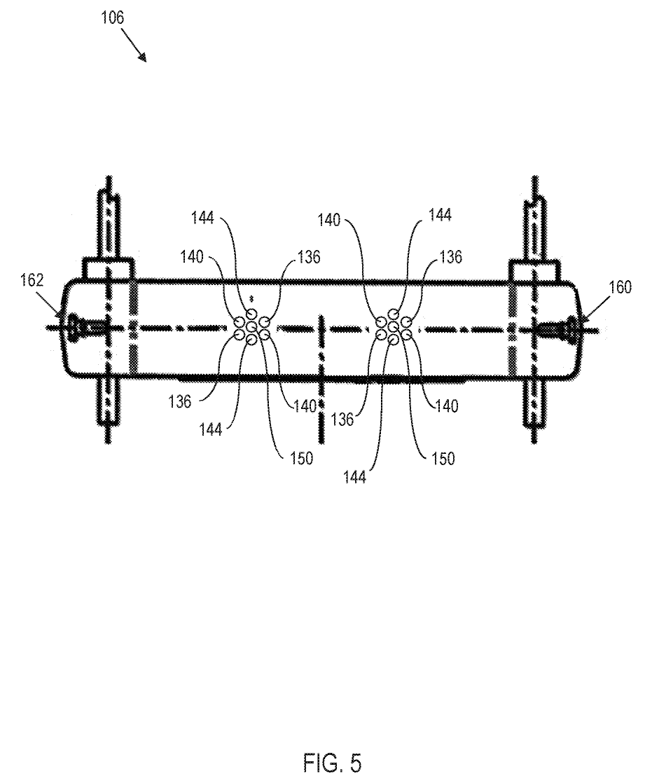

Turning now to FIG. 1, a device 100 for aiding patients with macular degeneration is shown. The device 100 includes a base and posts 104 rising from the base 102. While two posts 104 are shown in FIG. 1, any number of posts may be used. Further, the device 100 includes a visor 106 that includes a housing 108 that includes apertures 110 that correspond to the posts 104 such the visor 106 can be coupled to the base 102 via the apertures 110 and posts 104 while allowing the visor 106 to be repositioned. Further, the visor 106 includes an array of light-emitting devices (e.g., light-emitting diodes), as discussed below in reference to FIGS. 2 and 5.

The device 100 includes a crossbar 114 that spans two of the posts 104. Apertures 116 in the crossbar 114 allow the crossbar 114 to be coupled to the posts 104 of the base 102. At each aperture 116 of the crossbar 114, a fastener 118 is available for a user to tighten the crossbar 114 to the posts 104. By loosening the fasteners, the user can adjust the height of the crossbar 114 in relation to the base 102. Once a desired height is reached, the user can then tighten down the fasteners 118 to keep the crossbar 114 at that height. Further, the crossbar 114 includes a chinrest 120 that allows a patient to rest their chin on the device 100. The tightened fasteners 118 allow the patient to put weight on the chinrest 120 of the crossbar 114 without the crossbar 114 moving under the weight. Further, the visor 106 may be moved in relation to the crossbar 114 to allow for differences in a height between the chin and the eyes of the patient. While fasteners are not required on the visor 106, because no weight will be put on the visor 106, fasteners similar to the fasteners of the crossbar may be added to the visor 106.

Some embodiments of the device 100 include a power supply 124 that provides power to the visor 106. The power supply 124 may be a battery (e.g., lithium-ion, alkaline, rechargeable, non-rechargeable, etc.), alternating-current to direct-current converter, etc. Other embodiments of the device 100 do not include a power supply, as power may be supplied via an external device (e.g., a computer through a universal serial bus connection).

A controller (e.g., located in the visor 106, located in the base 102, external to the device, etc.) controls activation of the array of light-emitting devices, as discussed below. In some embodiments, the visor 106 includes a display to give information to a user or technician. The display may be a touchscreen or a display-only. In embodiments with a display-only display, a keypad or other input device may be implemented for the technician or patient to enter information, choose a treatment program, etc. In various embodiments, the visor 106 includes an audio device that relays audio information (e.g., commands of use, beeps, etc.) to the patient or technician (e.g., to start a new step in the processes described herein).

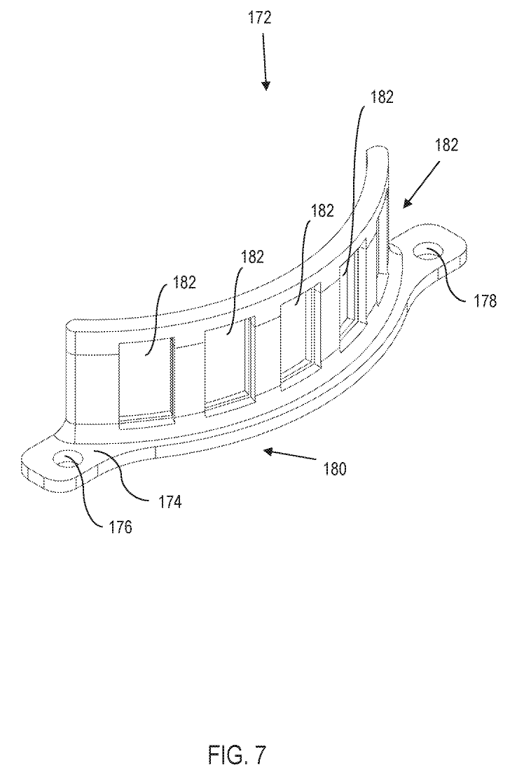

Moreover, various embodiments of the visor 106 include a communications port 126 that allows the visor to be part of a modular system that allows an ancillary device (172 FIG. 7) to be communicably coupled to the visor 106. For example, the ancillary device (172 FIG. 7) may be a device that includes several arrays or clusters of light-emitting devices that provide a light (visible, infrared, etc.) to a patient’s forehead, where operation of the ancillary device (172 FIG. 7) is provided by the controller in the visor 106.

Turning now to FIG. 2, a front view (i.e., the side that the patient accesses) of the device 100 is shown. The visor 106 includes an array of light-emitting devices 130 that includes a first cluster 132 of light-emitting devices and a second cluster of light-emitting devices 134. Each cluster 132, 134 includes a set of red (e.g., a wavelength of 625 nanometers) light-emitting devices 136, a set of green (e.g., wavelength between 505-525 nanometers) light-emitting devices 140, and a set of infrared (IR) light-emitting devices 144 (e.g., about 940 nanometers). Not all of the light-emitting devices have been marked for clarity. In various embodiments, the wavelengths of the three types of light-emitting devices are: 660nm for the light-emitting devices of the first color 136; 590nm for the light-emitting devices of the second color 140; and 850nm for the infrared (IR) light-emitting devices 144. In some embodiments, the light-emitting devices of the first color 136 include both 660nm and 625nm devices; the light-emitting devices of the second color include both 505nm and 590nm light-emitting devices; and the infrared (IR) light-emitting devices include both 940nm and 850nm light-emitting devices. Note that infrared light includes near infrared, short-wavelength infrared, mid-wavelength infrared, long-wavelength infrared, and far infrared.

As shown in FIG. 2, the first cluster 132 and second cluster 134 each include two light-emitting devices of the first color 136, two light-emitting devices of the second color 140, and two IR light-emitting devices 144. Further, In FIG. 2, those light-emitting devices are arranged such that the set of light-emitting devices of the first color includes two red light-emitting devices opposite each other around a central point 150. Further, the set of light-emitting devices of the second color includes two green (or amber) light-emitting devices opposite each other around the central point. Moreover, the set of IR light-emitting devices includes two IR light-emitting devices opposite each other around the central point.

However, other layouts of the light-emitting devices may be used. For example, the light-emitting devices may be laid out such that the set of light-emitting devices of the first color includes two red light-emitting devices opposite each other around a central point. Further, the set of light-emitting devices of the second color includes two green (or amber) light-emitting devices between the two light-emitting devices of the first color, on a single side of the central point. Moreover, the set of IR light-emitting devices includes two IR light-emitting devices between the two light-emitting devices of the first color, on a single side of the central point, opposite the two light-emitting devices of the second color. However, other layouts or numbers of light-emitting devices may be utilized.

In embodiments where the clusters 132, 143 include a central point 150, there may be another light-emitting device that indicates a warning that a procedure will commence or recommence soon. Thus, the patient will know to put their chin back on the chinrest 120 – as discussed below. Further, many embodiments of the device 100 include a wait-period indicator 152 (e.g., a light-emitting device) between the first cluster 132 and the second cluster 134 that indicates that the device 100 is in a wait period. Various embodiments of the device 100 include a diffusing lens 156 over the array of light-emitting devices.

The controller controls activation of the array of light-emitting devices such that the array of light-emitting devices is active for a first period of time. After the array of light-emitting devices is active for the first period of time, the array of light-emitting devices 130 is inactive for a second period of time. Then, after the array of light-emitting devices 130 is inactive for the second period of time, the array of light-emitting devices 130 is active for a third period of time. In many embodiments, the third period of time is equal to the first period of time. In numerous embodiments, the second period of time is equal to the first period of time. For example, the controller can control the array of light-emitting devices such that the first cluster 132 and second cluster 134 are active at the same time for five minutes. Then, the first cluster 132 and second cluster 134 are inactive for a period of five minutes. In embodiments with a wait-period indicator 152, the wait-period indicator 152 will be active during the time that five-minute period. Then, (in embodiments with the warning light), nearing the end of this period (e.g., 10 seconds left), the warning light at the central point 150 of the clusters 132, 134 will activate to let the patient know that the procedure will recommence. After the warning light, the controller controls the clusters 132, 134 to activate again for five minutes.

As used herein, when the array of light-emitting devices such that the array of light-emitting devices is active, both clusters 132, 134 may be active at the same time or the first cluster 132 activation may be active for a short period while the second cluster is inactive, then the second cluster 134 may be active for a short period while the first cluster is inactive. An activated cluster (or light-emitting device) may have continuous activation or intermittent activation at any duty cycle determined for proper treatment of macular degeneration.

For example, the first cluster and second cluster may be continuously active (which is really a 100% duty cycle) for five minutes during the first period of time. In another example, the first cluster and second cluster may be intermittently active with an 80% duty cycle (or other duty cycle) every few milliseconds (or other period of time). As a further example, the first cluster may be activated at a 75% duty cycle every millisecond for three seconds while the second cluster is off for three seconds; then the second cluster may be activated at a 75% duty cycle every millisecond for three seconds while the first cluster is off for three seconds; and this pattern repeats until the five-minute first period of time is over.

Moreover, all of the sets of light-emitting devices need not be active at the same time. Thus, the individual light-emitting devices of the clusters may be sequenced in any order (serial, parallel, etc.) while the cluster is active. For example, when the first cluster is active for three seconds, the first second may just be the light-emitting devices of the first color active at a 75% duty cycle every few milliseconds, then the second may be the light-emitting devices of the second color active at a 65% duty cycle every few milliseconds, and then the third second may be the IR light-emitting devices active at a 35% duty cycle every few milliseconds. Also, only one cluster may be activated for the entire process, because only one eye needs to be treated. Further, different intensities of light may be achieved by activating a subset of light-emitting devices for a color in a single cluster. For example, if a cluster of light-emitting devices includes two IR light-emitting devices, a low intensity may activate only one IR light emitting device, while a high intensity may activate both IR light-emitting devices.

By providing two clusters of light-emitting devices, the visor 106 is free of any mirrors or other ways to redirect light and allows both eyes to be treated at the same time.

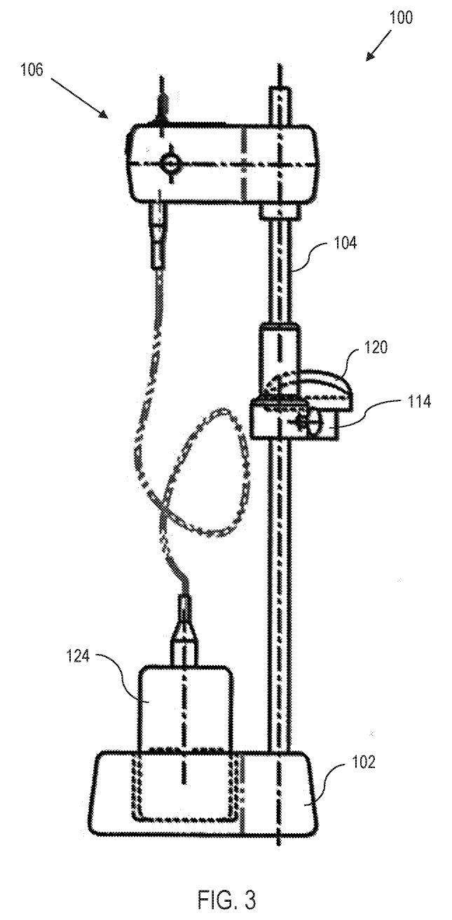

FIG. 3 is a side view of the device 100 of FIGS. 1 and 2, including the base 102, the posts 104, the visor 106, the crossbar 114 with the chinrest 120, and the power supply 124.

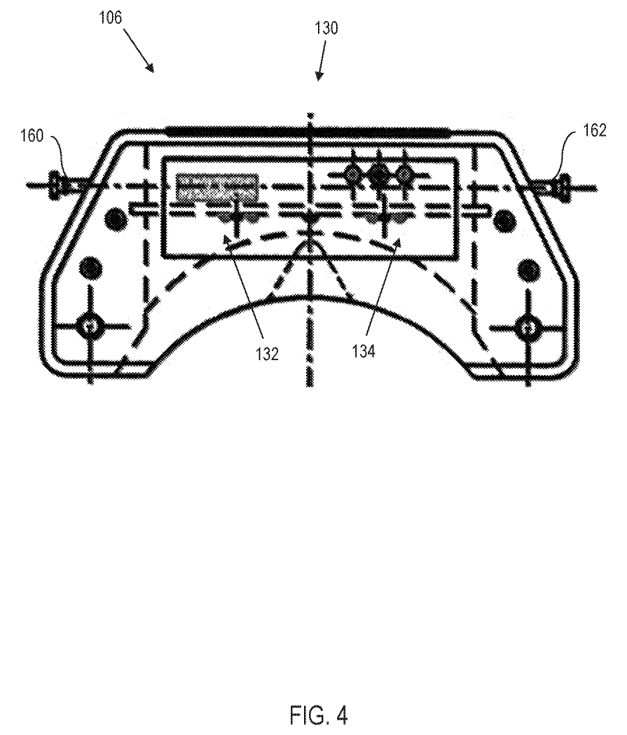

FIGS. 4-5 illustrate an embodiment that is just the visor 106 without the base or chinrest. Instead, the user holds the visor up to their eyes. The visor 106 includes an array of light-emitting devices 130 that includes a first cluster 132 of light-emitting devices and a second cluster of light-emitting devices 134. Each cluster 132, 134 includes a set of red (e.g., a wavelength of 625 nanometers) light-emitting devices 136, a set of green (e.g., wavelength between 505-525 nanometers) light-emitting devices 140, and a set of infrared (IR) (e.g., about 940 nanometers) light-emitting devices 144. In some embodiments, the clusters include red (e.g., 660nm), amber (590nm), and IR (850nm) light-emitting devices.

As shown in FIGS. 4-5, the first cluster 132 and second cluster 134 each include two light-emitting devices of the first color 136, two light-emitting devices of the second color 140, and two IR light-emitting devices 144. Further, In FIGS. 4-5, those light-emitting devices are arranged such that the set of light-emitting devices of the first color includes two red light-emitting devices opposite each other around a central point 150. Further, the set of light-emitting devices of the second color includes two green (or amber) light-emitting devices opposite each other around the central point. Moreover, the set of IR light-emitting devices includes two IR light-emitting devices opposite each other around the central point.

However, other layouts of the light-emitting devices may be used. For example, the light-emitting devices may be laid out such that the set of light-emitting devices of the first color includes two red light-emitting devices opposite each other around a central point. Further, the set of light-emitting devices of the second color includes two green (or amber) light-emitting devices between the two light-emitting devices of the first color, on a single side of the central point. Moreover, the set of IR light-emitting devices includes two IR light-emitting devices between the two light-emitting devices of the first color, on a single side of the central point, opposite the two light-emitting devices of the second color. However, other layouts or numbers of light-emitting devices may be utilized.

In embodiments where the clusters 132, 143 include a central point 150, there may be another light-emitting device that indicates a warning that a procedure will commence or recommence soon. Thus, the patient will know to put their chin back on the chinrest 120 – as discussed below. Further, many embodiments of the device 100 include a wait-period indicator 152 (e.g., a light-emitting device) between the first cluster 132 and the second cluster 134 that indicates that the device 100 is in a wait period. Various embodiments of the device 100 include a diffusing lens 156 over the array of light-emitting devices.

The controller controls activation of the array of light-emitting devices such that the array of light-emitting devices is active for a first period of time. After the array of light-emitting devices is active for the first period of time, the array of light-emitting devices 130 is inactive for a second period of time. Then, after the array of light-emitting devices 130 is inactive for the second period of time, the array of light-emitting devices 130 is active for a third period of time. In many embodiments, the third period of time is equal to the first period of time. In numerous embodiments, the second period of time is equal to the first period of time. For example, the controller can control the array of light-emitting devices such that the first cluster 132 and second cluster 134 are active at the same time for five minutes. Then, the first cluster 132 and second cluster 134 are inactive for a period of five minutes. In embodiments with a wait-period indicator (indicator 152, audio device, etc.), the wait-period indicator 152 will be active during that five-minute period. Then, (in embodiments with the warning light), nearing the end of this period (e.g., 10 seconds left), the warning light at the central point 150 of the clusters 132, 134 will activate to let the patient know that the procedure will recommence. After the warning light, the controller controls the clusters 132, 134 to activate again for five minutes.

As used herein, when the array of light-emitting devices such that the array of light-emitting devices is active, both clusters 132, 134 may be active at the same time or the first cluster 132 activation may be active for a short period while the second cluster is inactive, then the second cluster 134 may be active for a short period while the first cluster is inactive. An activated cluster (or light-emitting device) may have continuous activation or intermittent activation at any duty cycle determined for proper treatment of macular degeneration.

For example, the first cluster and second cluster may be continuously active (which is really a 100% duty cycle) for five minutes during the first period of time. In another example, the first cluster and second cluster may be intermittently active with an 80% duty cycle (or other duty cycle) every few milliseconds (or other period of time). As a further example, the first cluster may be activated at a 75% duty cycle every millisecond for three seconds while the second cluster is off for three seconds; then the second cluster may be activated at a 75% duty cycle every millisecond for three seconds while the first cluster is off for three seconds; and this pattern repeats until the five-minute first period of time is over.

Moreover, all of the sets of light-emitting devices need not be active at the same time. Thus, the individual light-emitting devices of the clusters may be sequenced in any order (serial, parallel, etc.) while the cluster is active. For example, when the first cluster is active for three seconds, the first second may just be the light-emitting devices of the first color active at a 75% duty cycle every few milliseconds, then the second second may be the light-emitting devices of the second color active at a 65% duty cycle every few milliseconds, and then the third second may be the IR light-emitting devices active at a 35% duty cycle every few milliseconds.

Many embodiments of the visor 106 (both solo and with the system of FIG. 1) include lateral adjustment knobs 160, 162 that allow a user to adjust a lateral spacing of each cluster of light-emitting devices, where a first adjustment knob 160 is coupled to the first cluster of light-emitting devices 132 and a second adjustment knob 162 is coupled to the second cluster of light-emitting devices 134. Specifically, the knobs 160, 162 can be pushed/pulled in or out of the visor for coarse adjustment of each of the cluster of light-emitting devices (or circuit boards on which the cluster of light-emitting devices are located). Once a coarse position is found, the user may then turn the knobs 160, 162 such that a threaded portion of the knobs goes into or out of the visor 106, which moves the associated cluster of light-emitting devices 132, 134 accordingly for a fine adjustment of the cluster of light-emitting devices 132, 134. Using the knobs 160, 162 for adjusting a lateral position of each of the cluster of light-emitting devices allows for the visor 106 to be used with people having different spacing between their eyes. The central point (150, FIG. 2) of each cluster of light-emitting devices may include a light-emitting device (e.g., a white light-emitting device or another color different than the colors of the light-emitting devices of the first and second colors) to assist the user in adjusting the cluster of light-emitting devices to align with pupils of the user's eyes. These light-emitting devices at the center point are not used during a treatment process – only as indications of different periods in the processes described herein.

Further, several embodiments of the visor 106 (both solo and with the system of FIG. 1) include circuitry to test the light-emitting devices of the clusters to determine if any of the light-emitting devices is operating out of specification. If any of the light-emitting devices are operating out of specification, then the process will be stopped if the process is using a high-intensity mode. On the other hand, if the device is operating in a lower intensity mode, then the light-emitting device that is operating out of specification will not be used for the low intensity mode, and another light-emitting device of the same color will be used instead.

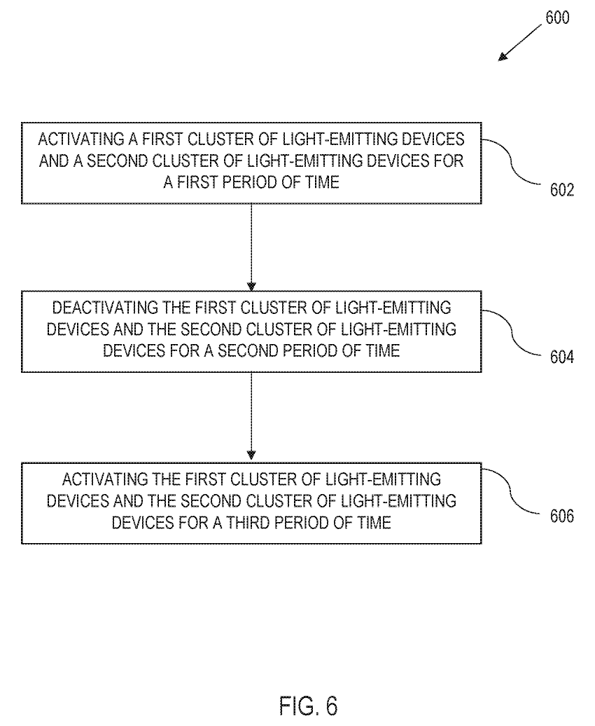

Turning now to FIG. 6, a process 600 for controlling a visor for aiding users with macular degeneration is shown. The visor includes an array of light-emitting devices that includes a first cluster of light-emitting devices and a second cluster of light-emitting devices. Each cluster includes a set of red (e.g., a wavelength of 625 nanometers) light-emitting devices, a set of green (e.g., wavelength between 505-525 nanometers) light-emitting devices, and a set of infrared (IR) (e.g., about 940 nanometers) light-emitting devices. In some embodiments, the clusters include red (e.g., 660nm), amber (590nm), and IR (850 nm) light-emitting devices.

At 602, a first cluster of light-emitting devices and a second cluster of light-emitting devices are activated for a first period of time. At 604, the first cluster of light-emitting devices and the second cluster of light-emitting devices are deactivated for a second period of time. At 606, the first cluster of light-emitting devices and the second cluster of light-emitting devices are activated again (after the second period of time) for a third period of time. In many embodiments, the third period of time is equal to the first period of time. In numerous embodiments, the second period of time is equal to the first period of time. For example, the controller can control the array of light-emitting devices such that the first cluster and second cluster are active at the same time for five minutes. Then, the first cluster and second cluster are inactive for a period of five minutes. In embodiments with a wait-period indicator, the wait-period indicator will be active during the time that five-minute period. Then, (in embodiments with the warning light), nearing the end of this period (e.g., ten seconds left), the warning light at the central point of the clusters will activate to let the patient know that the procedure will recommence. After the warning light, the controller controls the clusters to activate again for five minutes.

As used herein, when the array of light-emitting devices such that the array of light-emitting devices is active, both clusters may be active at the same time or the first cluster activation may be active for a short period while the second cluster is inactive, then the second cluster may be active for a short period while the first cluster is inactive. An activated cluster (or light-emitting device) may have continuous activation or intermittent activation at any duty cycle determined for proper treatment of macular degeneration.

For example, the first cluster and second cluster may be continuously active for five minutes during the first period of time. In another example, the first cluster and second cluster may be intermittently active with an 80% duty cycle (or other duty cycle) every few milliseconds (or other period of time). As a further example, the first cluster may be activated at a 75% duty cycle every millisecond for three seconds while the second cluster is off for three seconds; then the second cluster may be activated at a 75% duty cycle every millisecond for three seconds while the first cluster is off for three seconds; and this pattern repeats until the five-minute first period of time is over.

Moreover, all of the sets of light-emitting devices need not be active at the same time. Thus, the individual light-emitting devices of the clusters may be sequenced in any order (serial, parallel, etc.) while the cluster is active. For example, when the first cluster is active for three seconds, the first second may just be the light-emitting devices of the first color active at a 75% duty cycle every few milliseconds, then the second second may be the light-emitting devices of the second color active at a 65% duty cycle every few milliseconds, and then the third second may be the IR light-emitting devices active at a 35% duty cycle every few milliseconds. As an example, a 33% duty cycle for 30 milliseconds for each of the colors would be the first color LEDs on for ten milliseconds, off for 20 milliseconds; the second color LEDs on for ten milliseconds, off for 20 milliseconds; and the infrared LEDs on for ten milliseconds, off for 20 milliseconds.

Another example process includes activating the light-emitting devices of the second color (e.g., 590nm) and the IR light-emitting devices (e.g., 850) for a first period (e.g., thirty-five seconds) while the patient’s eye(s) are open at a desired duty cycle (e.g., 70% duty cycle, three times per second). Then, the patient closes their eyes, and the light-emitting devices of the first color (e.g., 660nm) are activated for a second time period (e.g., ninety seconds) at a duty cycle (e.g., 100% duty cycle – constantly). The process repeats those two time periods again for a full treatment.

Further, several embodiments of the visor 106 (both solo and with the system of FIG. 1) include circuitry to test the light-emitting devices of the clusters to determine if any of the light-emitting devices is operating out of specification. If any of the light-emitting devices is operating out of specification, then the process 600 will be stopped.

Turning to FIG. 7, as mentioned above, embodiments of the visor (both solo and with the system of FIG. 1) may be a modular visor and include a communications port 126 to allow the visor to communication with an ancillary device 172 . For example, studies have shown that light therapy using various waveforms of light on a patient’s forehead can help stroke victims, patients experiencing Chronic Traumatic Encephalopathy (CTE), and other issues. The ancillary device 172 includes a base 174 with apertures 176, 178 that align with the posts (104, FIG. 1) of the base (102, FIG. 1).

Further, an underside of the ancillary device 172 includes a communications port 180 that aligns with the communications port (126, FIG. 1) of the visor (106, FIG. 1).

Moreover, the ancillary device 172 includes modular ports 182, where modules with clusters of light-emitting devices may be added. Different types of clusters may be added. For example, the clusters may be similar to the clusters described above or they may include more or less light-emitting devices. Also, the clusters can include different colors of light-emitting devices (not just the red, green, amber, and IR described above) to match whichever light therapy may be required for a patient’s condition.

The communications ports allow the controller of the visor to control the clusters added to the ancillary device 172. Thus, the visor may be used for treating macular degeneration as discussed above or may be used for light therapy for other conditions (e.g., stroke, CTE, etc.) with the ancillary device.

Using the process above (where the first, second, and third periods of time are each five minutes), every day for five days and then repeating those five days every three months, wet-form macular degeneration in patients was shown to have decreased. As will be appreciated by one skilled in the art, aspects of the present disclosure may be embodied as a system, method or computer program product. Accordingly, aspects of the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present disclosure may take the form of a computer program product embodied in one or more computer readable storage medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or

more wires, a portable computer diskette, a hard disk, a random access memory (RAM), a read-only memory (ROM), an erasable programmable read-only memory (EPROM), Flash memory, an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device. A computer storage medium does not include propagating signals.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present disclosure may be written in any combination of one or more programming languages, including an object oriented programming language such as Java, Smalltalk, C++ or the like and conventional procedural programming languages, such as the "C" programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Network using a Network Service Provider).

Aspects of the present disclosure are described herein with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the Figures illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be

executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular embodiments only and is not intended to be limiting of the disclosure. As used herein, the singular forms "a", "an" and "the" are intended to include the plural forms as well, unless the context clearly indicates otherwise. It will be further understood that the terms "comprises" and/or "comprising," when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. Aspects of the disclosure were chosen and described in order to best explain the principles of the disclosure and the practical application, and to enable others of ordinary skill in the art to understand the disclosure for various embodiments with various modifications as are suited to the particular use contemplated.

What is claimed is:

1. A device for aiding patients with macular degeneration, the device comprising:
a base with posts;
a visor including:
a housing with apertures corresponding to the posts of the base such that the visor couples to the base via the apertures and the posts;
an array of light-emitting devices comprising:
a first cluster of the light-emitting devices including:
a first set of light-emitting devices of a first color;
a first set of light-emitting devices of a second color; and
a first set of infrared (IR) light-emitting devices; and
a second cluster of the light-emitting devices including:
a second set of light-emitting devices of the first color;
a second set of light-emitting devices of the second color; and
a second set of IR light-emitting devices; and
a controller that controls activation of the array of light-emitting devices such that:
the array of light-emitting devices is active for a first period of time;
after the array of light-emitting devices is active for the first period of time, the array of light-emitting devices is inactive for a second period of time; and
after the array of light-emitting devices is inactive for the second period of time, the array of light-emitting devices is active for a third period of time.

2. The device of claim 1, wherein the first set of light-emitting devices of the first color produce a wavelength of 625 nanometers.

3. The device of claim 1, wherein the first set of light-emitting devices of the second color produce a wavelength of 505-525 nanometers.

4. The device of claim 1, wherein the first set of IR light-emitting devices produce a wavelength of 940 nanometers.

5. The device of claim 1, wherein the first cluster of the light-emitting devices includes a central point and is arranged such that:
the first set of light-emitting devices of the first color includes two light-emitting devices of the first color opposite each other around the central point;
the first set of light-emitting devices of the second color includes two green light-emitting devices of the second color opposite each other around the central point; and
the first set of IR light-emitting devices includes two IR light-emitting devices opposite each other around the central point.

6. The device of claim 1, wherein the first cluster of the light-emitting devices includes a central point and is arranged such that:
the first set of light-emitting devices of the first color includes two light-emitting devices of the first color opposite each other around the central point;
the first set of light-emitting devices of the second color includes two light-emitting devices of the second color between the two light-emitting devices of the first color, on a single side of the central point; and
the first set of IR light-emitting devices includes two IR light-emitting devices between the two light-emitting devices of the first color, on a single side of the central point, opposite the two light-emitting devices of the second color.

7. The device of claim 1, wherein the first cluster of the light-emitting devices includes a central point and a warning light-emitting device at the central point.

8. The device of claim 1, wherein a wait-period indicator is between the first cluster of light-emitting devices and the second cluster of light-emitting devices.

9. The device of claim 1, wherein the third period of time is equal to the first period of time.

10. The device of claim 9, wherein the second period of time is equal to the first period of time.

11. The device of claim 1, wherein the controller is housed inside the visor.

12. The device of claim 11, wherein the visor includes a communications port, and the system includes:
an ancillary device that couples to the visor via the communications port of the visor and a communications port of the ancillary device, wherein the ancillary device includes light-emitting devices and is controlled by the controller of the visor.

13. The device of claim 1, wherein both the first cluster of light-emitting devices and the second set of light-emitting devices are activated at the same time during the first period of time.

14. The device of claim 1, wherein both the first cluster of light-emitting devices and the second set of light-emitting devices are activated at opposite times during the first period of time.

15. A process for controlling a visor for aiding users with macular degeneration, the process comprising:
activating a first cluster of light-emitting devices and a second cluster of light-emitting devices for a first period of time, wherein:
the first cluster of light-emitting devices includes a first set of light-emitting devices of a first color, a first set of light-emitting devices of a second color, and a first set of infrared (IR) light-emitting devices; and
the second cluster of light-emitting devices includes a second set of light-emitting devices of the first color, a second set of light-emitting devices of the second color, and a second set of infrared (IR) light-emitting devices;
deactivating the first cluster of light-emitting devices and the second cluster of light-emitting devices for a second period of time; and
activating the first cluster of light-emitting devices and the second cluster of light-emitting devices for a third period of time.

16. The process of claim 15, wherein the third period of time is equal to the first period of time.

17. The process of claim 16, wherein the second period of time is equal to the first period of time.

18. The process of claim 15, wherein both the first cluster of light-emitting devices and the second set of light-emitting devices are activated at opposite times during the first period of time.

19. The process of claim 15, wherein both the first cluster of light-emitting devices and the second set of light-emitting devices are activated at the same time during the first period of time.

20. A visor for use in aiding patients with macular degeneration, the visor comprising:
an array of light-emitting devices comprising:
a first cluster of the light-emitting devices including:
a first set of light-emitting devices of a first color;
a first set of light-emitting devices of a second color; and
a first set of infrared (IR) light-emitting devices; and
a second cluster of the light-emitting devices including:
a second set of light-emitting devices of the first color;
a second set of light-emitting devices of the second color; and
a second set of IR light-emitting devices; and
a controller that controls activation of the array of light-emitting devices such that:
the array of light-emitting devices is active for a first period of time;
after the array of light-emitting devices is active for the first period of time, the array of light-emitting devices is inactive for a second period of time; and
after the array of light-emitting devices is inactive for the second period of time, the array of light-emitting devices is active for a third period of time.
